(19) United States
(12) Patent Application Publication
(10) Pub. No.: US 2016/0129148 A1
(43) Pub. Date: May 12, 2016

(54) Nano-calcium phosphate-coated polymethylmethacrylate-based co-polymer and coating process of the same

(71) Applicant: NANO AND ADVANCED MATERIALS INSTITUTE LIMITED, HONG KONG (HK)

(72) Inventors: Chi Tak WONG, New Territories (HK); Sau Kuen Connie Kwok, New Territories (HK)

(21) Appl. No.: 14/933,019

(22) Filed: Nov. 5, 2015

Related U.S. Application Data
(60) Provisional application No. 62/123,108, filed on Nov. 7, 2014.

(51) Int. Cl. A61L 24/06; A61L 24/00; A61L 24/02

(52) U.S. Cl. CPC A61L 24/06; A61L 24/02; A61L 24/0015; A61L 2420/02; A61L 2400/12; A61L 2300/606; A61L 2300/112

(57) ABSTRACT

The present invention relates to a method for coating polymethylmethacrylate (PMMA)-based co-polymer beads with nano-calcium phosphate. The method includes synthesizing the PMMA-based co-polymer beads containing hydroxyl pendant group, reacting the calcium salt and phosphate solution with the hydroxyl pendant group on the PMMA-based co-polymer beads, and thickening of the nano-calcium phosphate coating on the PMMA-based co-polymer beads.

### CROSS REFERENCE TO RELATED APPLICATION

The present invention claims the benefits and advantages of a U.S. provisional application Ser. No. US 62/123,108, filed on Nov. 5, 2014, entitled “Nano-calclum phosphate-coated polymethylmethacrylate-based co-polymer”, of which an entirety is incorporated here for reference.

### TECHNICAL FIELD

The present invention relates to bioactive polymethylmethacrylate (PMMA)-based co-polymers, processes for their preparation and their use as bioactive filler for use in orthopaedic, dental, polymer and chemical applications.

### BACKGROUND

Bone filler is a material which is used to fill or replace bones or teeth. Acrylic bone cement containing a solid phase with PMMA and a liquid phase with methyl methacrylate monomer can be injected and hardened in a human body. It finds wide applications in arthroplasty fixation and vertebroplasty.

PMMA itself is bio-inert and cannot bond to bone. Alternatively, calcium phosphate (CaP), for example, can be added to offer bioactivity to PMMA. CaP is a family of minerals which contains calcium cations and phosphate anions. CaP is biocompatible, biodegradable, and is the main mineral component of bones. It has shown to improve bone bonding, cell adhesion and new bone formation.

### SUMMARY OF THIS DISCLOSURE

The present invention can provide methods to apply a nano-calcium phosphate coating on polymethylmethacrylate (PMMA)-based co-polymer beads. Due to the existence of hydroxyl pendant group on the PMMA-based co-polymer, calcium phosphate can be formed by chemical reaction and accumulated on the co-polymer over time.

In one aspect, there is provided a method for coating PMMA-based co-polymer beads with nano-calcium phosphate. The method can include synthesizing the PMMA-based co-polymer beads containing multiple hydroxyl pendant groups, reacting a calcium salt and a phosphate solution with the hydroxyl pendant groups on the PMMA-based co-polymer beads, and thickening of the nano-calcium phosphate coating on the PMMA-based co-polymer beads.

In another aspect, there is provided a nanoparticle comprising a PMMA-based co-polymer bead coated with nano-calcium phosphate. The nanoparticle can have a diameter of about 10 nm to 1000 nm. The nanoparticle may be useful for bone cement, bone filler or teeth filling.

In yet another aspect, there is provided a microparticle comprising a polymethylmethacrylate-based co-polymer bead coated with calcium phosphate. The microparticle can have a diameter of about 1 μm to 100 μm. The microparticle may be useful for bone cement, bone filler or teeth filling.

The nano-calcium phosphate coated PMMA-based co-polymer beads can be used as a major component to offer bioactivity to bone cement. The advantages of this invention include chemical bonding of bioactive calcium phosphate directly on the PMMA-based co-polymer beads, control of coating thickness from nanometer to submicron size, and further polymerization of the nano-calcium phosphate coated co-polymer beads is possible.

### BRIEF DESCRIPTION OF THE DRAWINGS

Following detailed descriptions of respective embodiments in this disclosure can be understood better when combining with these figures, in which the same structure is represented by the same reference sign. In the figures:

FIG. 1 illustrates coating nano-calcium phosphate on PMMA-based co-polymer bead in this disclosure;

FIG. 2 shows an SEM image for PMMA-based beads (P);

FIG. 3 shows an SEM image for PMMA-based beads (PH-1);

FIG. 4 shows an SEM image for PMMA-based beads (PH-0.5);

FIG. 5 shows an SEM image for nano-calcium phosphate coated PMMA-based beads (PH-0.5) mixed for 1 day;

FIG. 6 shows an SEM image for nano-calcium phosphate coated PMMA-based beads (PH-0.5) mixed for 2 days;

FIG. 7 shows an SEM image for nano-calcium phosphate coated PMMA-based beads (PH-0.5) mixed for 3 days;

FIG. 8 shows EDX analysis on the nano-calcium phosphate coated PMMA-based beads in FIG. 5 for 1 day;

FIG. 9 shows EDX analysis on the nano-calcium phosphate coated PMMA-based beads in FIG. 6 for 2 days;

FIG. 10 shows EDX analysis on the nano-calcium phosphate coated PMMA-based beads in FIG. 7 for 3 days;

FIG. 11 shows setting properties of bioactive bone cement;

FIG. 12 shows an SEM image of bioactive bone cement after immersed in DMEM for 3 days; and

FIG. 13 shows EDX analysis on an apatite layer after immersed in DMEM for 3 days.

### DETAILED DESCRIPTION

The present invention can provide methods to apply a nano-calcium phosphate coating on polymethylmethacrylate (PMMA)-based co-polymer beads to form a bioactive PMMA-based co-polymer. Due to the existence of hydroxyl pendant group on the PMMA-based co-polymer, calcium phosphate can be formed by chemical reaction and accumulated over time. The nano-calcium phosphate coated PMMA-based co-polymer beads can be used as a major component to offer bioactivity to bone cement.

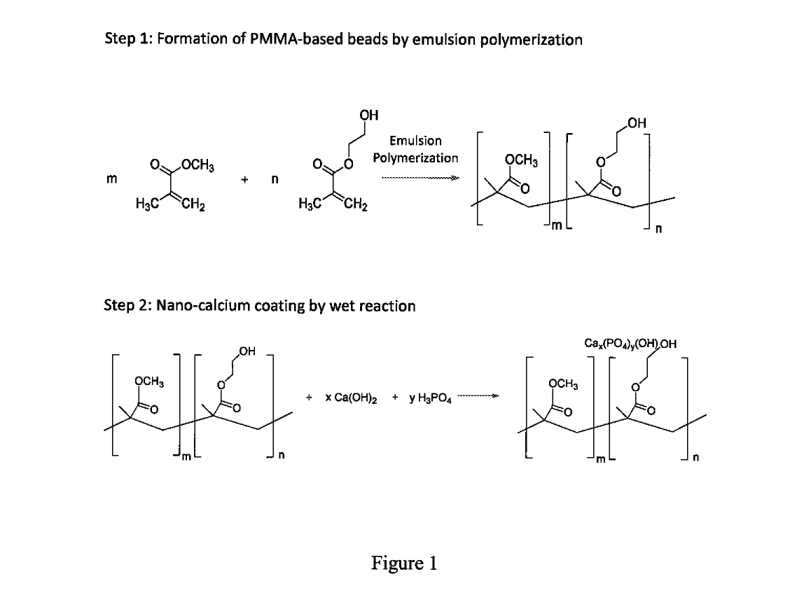

FIG. 1 illustrates an embodiment of a coating method in the present invention. The method can include synthesizing the PMMA-based co-polymer beads containing hydroxyl pendant group, reacting a calcium salt and a phosphate solution with the hydroxyl pendant group on the PMMA-based co-polymer beads to form the nano-calcium phosphate, and thickening of the nano-calcium phosphate coating on the PMMA-based co-polymer beads.

PMMA-based co-polymer beads containing hydroxyl pendant group can be synthesized by emulsion polymerization. Emulsion polymerization is a type of radical polymerization that starts with an emulsion incorporating water, monomer and surfactant. Monomers can be any compound contains one or more methacrylate functional groups (which can also be called as the methacrylate-based monomer). Monomers can be selected but not limited from a group of methyl methacrylate (MMA), 2-hydroxyethyl methacrylate (HEMA), 3-(acryloyloxy)-2-hydroxypropyl methacrylate, allyl methacrylate, 2-[3-(2H-benzotriazol-2-yl)-4-hydroxyphenyl]ethyl methacrylate, butyl methacrylate, 3-chloro-2-hydroxypropyl methacrylate, ethyl methacrylate, ethylene glycol dimethacrylate, glycidyl methacrylate, hydroxybutyl methacrylate, hydroxyethyl acrylate, hydroxymethyl methacrylate, hydroxypropyl methacrylate, 2-hydroxy-3-{3-[2,4,6,8-tetramethyl-4,6,8-tris(propyl glycidyl ether)-2-cyclotetrasiloxanyl]propoxy}propyl methacrylate, 2-methoxyethyl methacrylate, and triethylene glycol dimethacrylate. Potassium peroxodisulfate can be used to initiate the polymerization. In the water solution, the potassium peroxodisulfate dissociates to give radicals. Methyl methacrylate and other methacrylate-based monomer react by free radical polymerization to give the PMMA-based co-polymer beads.

The PMMA-based co-polymer beads shall contain a hydroxyl pendant group in order to react with the calcium salt and the phosphate solution. Correspondingly, at least some of the monomers used during the emulsion polymerization shall have a hydroxyl group. Hydroxyapatite, a type of calcium phosphate, can be formed by wet reaction based on the following stoichiometric equation:

10Ca(OH)₂+6H₃PO₄→Ca₁₀(PO₄)₆(OH)₂+18H₂O.

The hydroxyl pendant group on the PMMA-based beads can provide a site to replace a OH functional group on the Ca₁₀(PO₄)₆(OH)₂ during reaction of calcium hydroxide with phosphoric acid. The hydroxyl group during formation of the calcium phosphate can be replaced by the hydroxyl pendant group on the PMMA-based co-polymer beads. Calcium cations and phosphate anions can continue to accumulate on the hydroxyl pendant group on the PMMA-based co-polymer beads. Finally, a nano-calcium phosphate coating is formed on the PMMA-based co-polymer beads, where the nano-calcium phosphate coating is bonded to the PMMA-based co-polymer beads through chemical bonding.

The nano-calcium phosphates are formed by the chemical reaction with the hydroxyl pendant group on the PMMA-based co-polymer beads in the present invention. The nano-calcium phosphate has a diameter of about 10 nm to 1000 nm. The nano-calcium phosphate coated PMMA-based co-polymer bead can be a nanoparticle that has a diameter of about 10 nm to 1000 nm. The nano-calcium phosphate coating can be accumulated over time and a microparticle can then be formed. The microparticle comprising the nano-calcium phosphate coated PMMA-based co-polymer bead can have a diameter of about 1 μm to 100 μm.

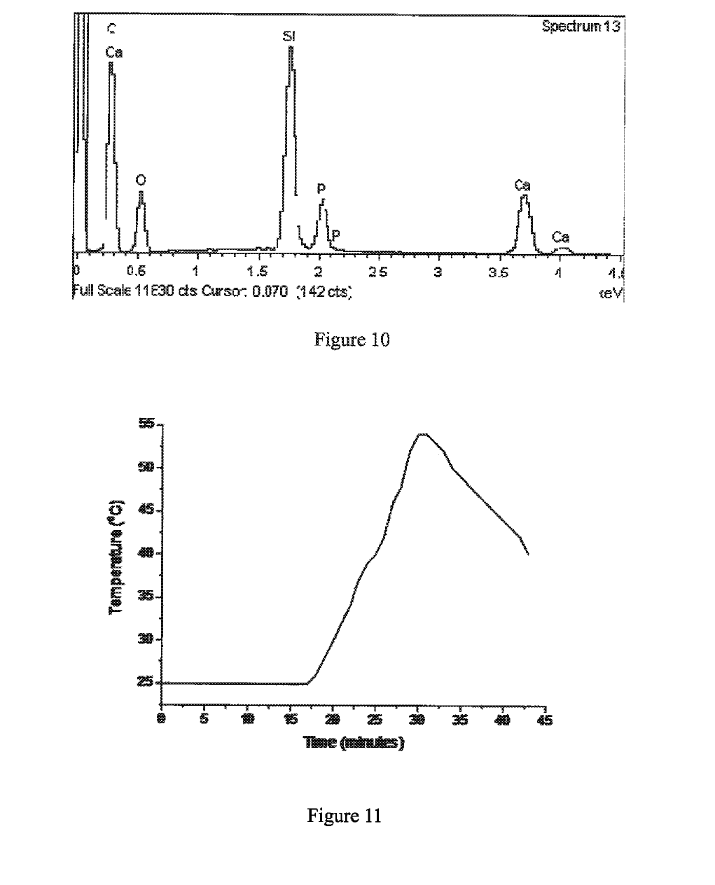

The nano-calcium phosphate coated PMMA-based co-polymer beads can be used as a major component to offer bioactivity to bone cement. Further polymerization of the nano-calcium phosphate coated PMMA-based co-polymer beads is possible. FIGS. 10 and 11 show the evidence that apatite can be formed on a bone cement pellet prepared using the nano-calcium phosphate coated PMMA-based co-polymer beads, indicating the bioactivity of this material. The nano-calcium phosphate coated PMMA-based co-polymer beads can be used in orthopaedic, dental, polymer and chemical applications.

Below the present invention will be described in detail with reference to various examples and reference drawings.

### EXAMPLE 1

### Synthesis of PMMA-based Co-polymer Beads Containing Hydroxyl Pendant Group

PMMA-based co-polymer beads containing hydroxyl pendant group(s) were synthesized by emulsion polymerization. In general, monomers (methyl methacrylate or other methacrylate monomer with hydroxyl pendant group such as 2-hydroxyethyl methacrylate), distilled water, and potassium peroxodisulfate were mixed and heated at 80° C. for 3 hours under nitrogen atmosphere. Then, the mixture was centrifuged and washed with distilled water. The PMMA-based beads were dried using a freeze dryer.

The types of PMMA-based heads synthesized are summarized in Table 1.

| Types | Monomers used | Product Code | Molar Ratio |
|---|---|---|---|
| 1 | Methyl methacrylate | P | 1 |
| 2 | Methyl methacrylate (MMA): | PH-1 | 1:1 |
| | 2-hydroxyethyl methacrylate | | |
| | (HEMA) | | |
| 3 | Methyl methacrylate (MMA): | PH-0.5 | 2:1 |
| | 2-hydroxyethyl methacrylate | | |
| | (HEMA) | | |

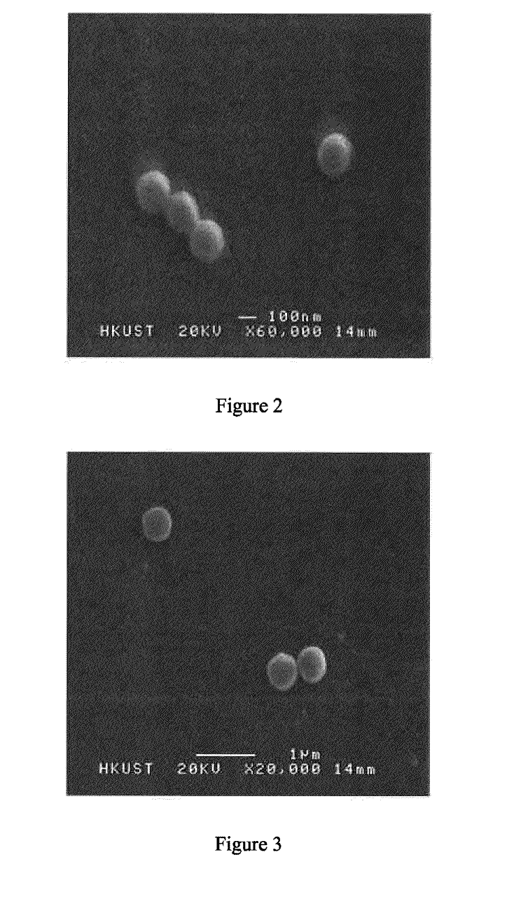

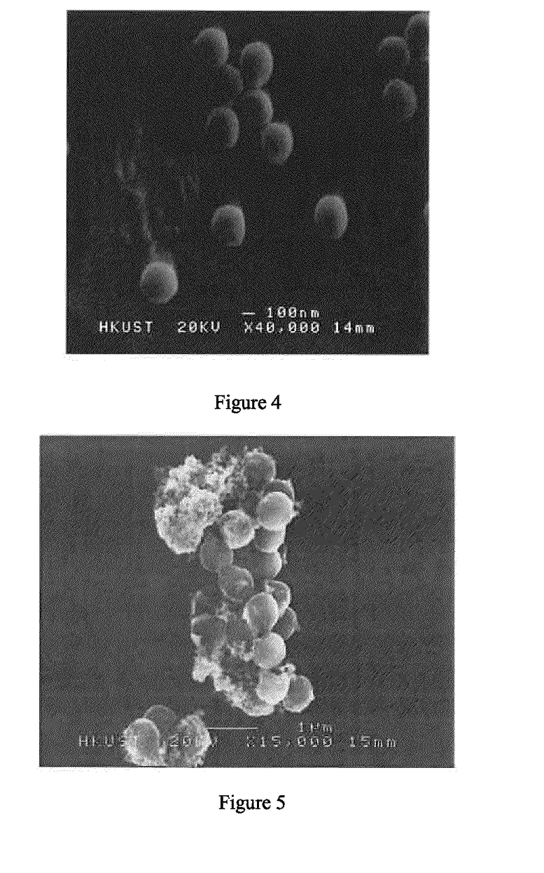

The PMMA-based beads (P, PH-1 and PH-0.5) were characterized using scanning electron microscopy (SEM). The SEM images of the PMMA-based beads are shown in FIGS. 2-4.

### EXAMPLE 2

### Nano-calcium Phosphate Coating on PMMA-based Co-polymer Beads

PMMA-based beads (PH-0.5) and calcium hydroxide were mixed with distilled water in a beaker. Phosphoric acid was added into the reaction mixture dropwisely using a syringe pump (Rate: 0.139 ml/hr). After all the phosphoric acid was added, the reaction mixture was mixed for 1, 2 and 3 days, respectively. After the mixing was completed, the mixture was centrifuged and freeze dried.

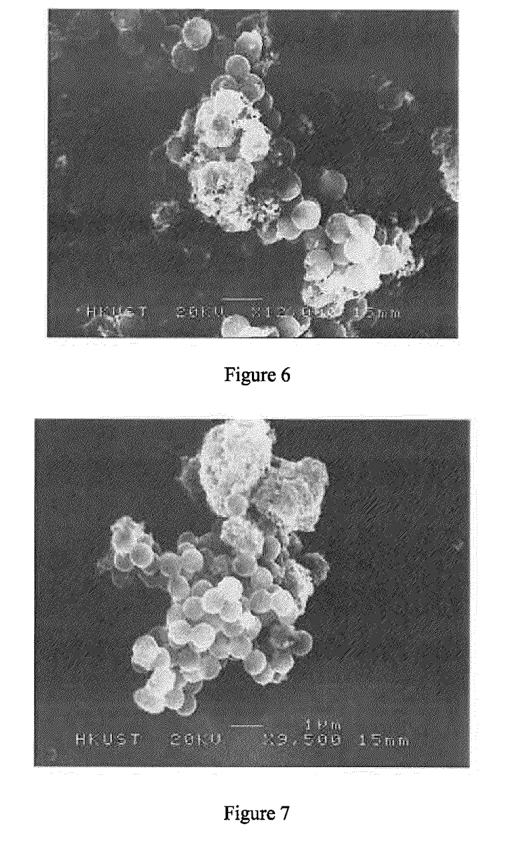

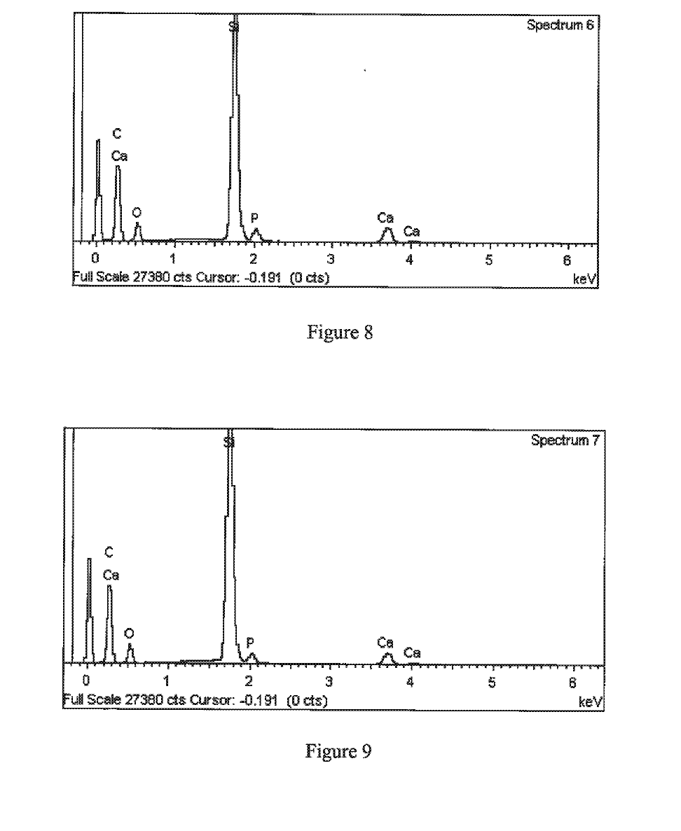

The nano-calcium phosphate coated PMMA-based co-polymer beads were observed by scanning electron microscopy (SEM) and analyzed by energy dispersive X-ray (EDX) analysis. The nano-calcium phosphate coated PMMA-based beads were fixed on a Si wafer and coated with carbon for the SEM observation. FIGS. 5-7 show the SEM images of reaction products mixed for 1, 2 and 3 days, respectively. The calcium phosphate coating was thickened with prolonged mixing periods. Energy dispersive X-ray (EDX) analysis was performed on the nano-calcium coated PMMA-based beads for the 1, 2 and 3 days samples as shown in FIGS. 8-10, indicating Ca and P signals. Existence of calcium and phosphate on the PMMA-based beads are confirmed.

Bioactivity of the nano-calcium phosphate coated PMMA-based co-polymer beads is confirmed by apatite formation test. Herein, setting parameters of bioactive bone cement formed by the nano-calcium phosphate coated PMMA-based co-polymer beads was first determined. The bioactive bone cement was formed using nano-calcium phosphate coated PMMA-based co-polymer beads (36 g), methyl methacrylate monomer (36 g), benzoyl peroxide (1.04 g) and N,N-dimethyl-p-toluidine (1.30 g). Also, some other formulations can be used to obtain the bioactive bone cement that contains the nano-calcium phosphate coated PMMA-based co-polymer beads, so that different setting properties are achieved corresponding to the specific formulations of the bioactive bone cement.

All the compounds were mixed in a beaker and then temperatures were measured using a thermometer. A setting graph of the bioactive bone cement is shown in FIG. 11, where it is found that the bioactive bone cement started to set at 17 minutes after mixing all the compounds, and a maximum temperature measured was 54° C. which was lower than commercial PMMA bone cement.

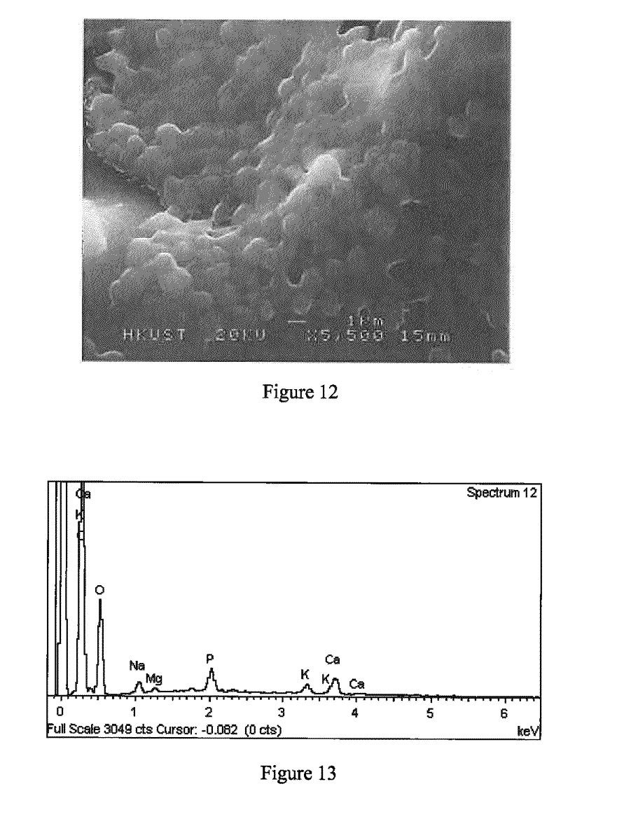

Also, those compounds were mixed and set in a mold which formed pellets (Diameter: 9.5 mm; Height: 5 mm). After setting, the bone cement was taken out. The pellets (n=6) were kept in 20 ml vials containing 10 ml of Dulbecco's Modified Eagle's Medium (DMEM). The vials were kept in a 37 ° C. water bath for 3 days. After 3 days, the pellets were taken out and washed with distilled water and then air dried. Pure PMMA pellets prepared by the afore-described PMMA-based beads (P) were taken as the control. The bioactive bone cement pellets were observed using scanning electron microscopy. The SEM image is shown in FIG. 12. Layer of apatite was formed on top of the bioactive bone cement but not observed in pure PMMA cement. EDX analysis was performed on the apatite layer and the result is shown in FIG. 13. Apatite formation was confirmed. In PMMA control group, no apatite was observed and calcium and phosphorus signals could not be detected by EDX analysis. The apatite can indicate the bioactivity of the nano-calcium phosphate coated PMMA-based co-polymer beads.

1. A bioactive polymethylmethacrylate (PMMA)-based co-polymer, comprising PMMA-based co-polymer beads and nano-calcium phosphate coated on surfaces of the PMMA-based co-polymer beads.

2. The PMMA-based co-polymer of claim 1, wherein the nano-calcium phosphate is coated onto the PMMA-based co-polymer beads through chemical bonding.

3. The PMMA-based co-polymer of claim 2, wherein the PMMA-based co-polymer beads contains multiple hydroxyl pendant groups; the nano-calcium phosphate is formed by chemical reaction with the multiple hydroxyl pendant groups on the PMMA-based co-polymer beads.

4. The PMMA-based co-polymer of claim 2, wherein the PMMA-based co-polymer beads contains multiple hydroxyl pendant groups; one or more of the multiple hydroxyl pendant groups act(s) as one hydroxyl of the nano-calcium phosphate to bond the nano-calcium phosphate to the surface of the PMMA-based co-polymer beads.

5. The PMMA-based co-polymer of claim 1, wherein the PMMA-based co-polymer beads are prepared by emulsion polymerization method from methacrylate-based monomers.

6. The PMMA-based co-polymer of claim 1, wherein the PMMA-based co-polymer is a nanoparticle having a diameter of substantially 10-1000 nm.

7. The PMMA-based co-polymer of claim 1, wherein the PMMA-based co-polymer is a microparticle having a diameter of substantially 1-100 μm.

8. The PMMA-based co-polymer of claim 1, wherein the nano-calcium phosphate has a diameter of substantially 10-1000 nm.

9. A method for coating polymethylmethacrylate (PMMA)-based co-polymer beads with nano-calcium phosphate, comprising:
synthesizing the PMMA-based co-polymer beads containing multiple hydroxyl pendant groups; and
reacting a calcium salt and a phosphate solution with the multiple hydroxyl pendant groups on the PMMA-based co-polymer beads to form a nano-calcium phosphate coating on the PMMA-based co-polymer beads.

10. The method of claim 9, wherein the PMMA-based co-polymer beads are produced by emulsion polymerization method, and monomers containing one or more methacrylate functional groups are used for the emulsion polymerization method.

11. The method of claim 10, wherein the monomers are to be selected but not limited from a group of methyl methacrylate, 2-hydroxyethyl methacrylate, 3-(acryloyloxy)-2-hydroxypropyl methacrylate, allyl methacrylate, 2-[3-(2H-benzotriazol-2-yl)-4-hydroxyphenyl]ethyl methacrylate, butyl methacrylate, 3-chloro-2-hydroxypropyl methacrylate, ethyl methacrylate, ethylene glycol dimethacrylate, glycidyl methacrylate, hydroxybutyl methacrylate, hydroxyethyl acrylate, hydroxymethyl methacrylate, hydroxypropyl methacrylate, 2-hydroxy-3-{3-[2,4,6,8-tetramethyl-4,6,8-tris(propyl glycidyl ether)-2-cyclotetrasiloxanyl]propoxy}propyl methacrylate, 2-methoxyethyl methacrylate, and triethylene glycol dimethacrylate.

12. The method of claim 9, wherein forming the nano-calcium phosphate coating on the PMMA-based co-polymer beads comprises:
dissolving the PMMA-based co-polymer beads and the calcium salt with a solvent to obtain a first mixture;
adding the phosphate solution into the first mixture to obtain a reaction mixture; and
continuously mixing the reaction mixture and centrifuging the reaction mixture to separate the PMMA-based co-polymer beads coated with the nano-calcium phosphate from the solvent.

13. The method of claim 9, wherein the nano-calcium phosphate has a diameter of substantially 10-1000 nm.

14. The method of claim 9, furthering comprising thickening the nano-calcium phosphate coating on the PMMA-based co-polymer beads.

15. A nanoparticle comprising polymethylmethacrylate-based co-polymer beads coated with nano-calcium phosphate, wherein the nanoparticle is obtained by a method of claim 9, and the nanoparticle has a diameter of substantially 10-1000 nm.

16. A microparticle comprising polymethylmethacrylate-based co-polymer beads coated with nano-calcium phosphate, wherein the microparticle is obtained by a method of claim 9, and the microparticle has a diameter of substantially 1-100 μm.

17. Use of the nanoparticle of claim 15 in bone cement, bone filler or teeth filling to offer bioactivity.

18. Use of the microparticle of claim 16 in bone cement, bone filler or teeth filling to offer bioactivity.

19. Use of the polymethylmethacrylate-based co-polymer of claim 1 in bone cement, bone filler or teeth filling to offer bioactivity.

20. Use of nano-calcium phosphate coated polymethylmethacrylate-based co-polymer beads in bone cement, bone filler or teeth filling to offer bioactivity, wherein the nano-calcium phosphate coated PMMA-based co-polymer beads are obtained by a method of claim 9.
